(19) United States
(12) Patent Application Publication
(10) Pub. No.: US 2010/0209932 A1
(43) Pub. Date: Aug. 19, 2010

(54) MicroRNA and Messenger RNA Detection On Arrays

(75) Inventor: EUGENE G. SPIER, Los Altos, CA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/706,627

(22) Filed: Feb. 16, 2010

Related U.S. Application Data
(63) Continuation of application No. 11/562,359, filed on Nov. 21, 2006, now abandoned.
(60) Provisional application No. 60/781,208, filed on Mar. 10, 2006.
(60) Provisional application No. 60/790,472, filed on Apr. 7, 2006.
(60) Provisional application No. 60/800,376, filed on May 15, 2006.

(51) Int. Cl. C12Q 1/68

(52) U.S. Cl. 435/6

(57) ABSTRACT

The present teachings provide methods for reverse transcribing, and detecting, a plurality of small nucleic acids such as micro RNAs, from the same reaction mixture as a plurality of messenger RNAs. High levels of multiplexing are provided by the use of a plurality of zip-coded stem-loop reverse transcription primers, along with an oligo-dT-promoter-containing reverse transcription primer, in the same reverse transcription reaction mixture. The resulting products can be amplified in an in vitro transcription reaction, and detected on a solid support such as an array. The present teachings also provide compositions, kits, and devices for performing and detecting the reverse transcription reactions described herein.

### CROSS REFERENCE TO RELATED APPLICATIONS

This application is a continuation of U.S. application Ser. No. 11/562,359, filed Nov. 21, 2006, which claims a priority benefit under 35 U.S.C. §119(e) from U.S. Patent Application No. 60/781,208, filed Mar. 10, 2006, U.S. Patent Application No. 60/790,472, filed Apr. 7, 2006 and U.S. Patent Application No. 60/800,376, filed May 15, 2006, which are incorporated herein by reference.

### FIELD

The present teachings are in the field of molecular and cell biology, specifically in the field of multiplexed detection of messenger RNAs along with short nucleic acids such as micro RNAs.

### BACKGROUND

Numerous fields in molecular biology require the identification of target polynucleotide sequences. Reverse transcription and amplification are two frequently used procedures employed to query the identity of target polynucleotides. The increasing amount of sequence information available to scientists in the post-genomics era has produced an increased need for rapid, reliable, low-cost, high-throughput, sensitive, and accurate methods to query complex nucleic acid samples. Methods of defining and characterizing cells have been hindered by robust amplification technologies, as well as the molecular complexity of conventionally analyzed molecules such as messenger RNA. Micro RNAs are a recently discovered class of molecules that offer great promise in understanding cell function. However, quantitative analysis of micro RNA has been hindered by their relatively short size.

### SUMMARY

The present teachings provide a method of detecting a plurality of short target nucleic acids, and a plurality of messenger RNAs on a same solid support, wherein each short target nucleic acid is 18-25 nucleotides in length, said method comprising; contacting the plurality of different short target nucleic acids with a plurality of target-specific stem-loop reverse transcription primers, wherein each of the plurality of stem-loop reverse transcription primers comprises a 3′ target-specific portion, a zip-code stem, and a promoter; contacting, in the same reaction mixture as the short target nucleic acids, a plurality of messenger RNAs with an oligo-dT-promoter-containing reverse transcription primer; extending the plurality of reverse transcription primers and the oligo-dT-promoter-containing reverse transcription primer in a reverse transcription reaction to form a collection of reverse transcription products; amplifying the reverse transcription products in an in vitro transcription reaction comprising an enzyme corresponding to the promoter, to form a plurality of in vitro transcription products; and, detecting the plurality of in vitro transcription products on the same solid support. Reaction compositions, kits, and devices are also provided.

### BRIEF DESCRIPTION OF THE DRAWINGS

The skilled artisan will understand that the drawings, described below, are for illustration purposes only. The drawings are not intended to limit the scope of the present teachings in any way.

FIG. 1 depicts certain aspects of various compositions according to some embodiments of the present teachings.

FIG. 2 depicts certain aspects of various compositions according to some embodiments of the present teachings.

FIG. 3 depicts certain aspects of various compositions according to some embodiments of the present teachings.

### DESCRIPTION OF EXEMPLARY EMBODIMENTS

Aspects of the present teachings may be further understood in light of the following examples, which should not be construed as limiting the scope of the present teachings in any way. The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. For example, “a target-specific stem-loop reverse transcription primer” means that more than one target-specific stem-loop reverse transcription primer can, but need not be present; for example but without limitation, one or more copies of a particular primer species, as well as one or more versions of a particular primer type. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention.

### SOME DEFINITIONS

As used herein, the term “short target nucleic acid” refers to a polynucleotide sequence that is sought to be amplified and quantitated, and that is between 18 and 25 nucleotides in length. The short target nucleic can be obtained from any source, and can comprise any number of different compositional components. For example, the target nucleic acid can be DNA, RNA, transfer RNA, siRNA, and can comprise nucleic acid analogs or other nucleic acid mimics, though typically the short target nucleic acids will be micro RNAs (miRNAs) and other short RNAs. The target can be methylated, non-methylated, or both. The target can be bisulfite-treated and non-methylated cytosines converted to uracil. Further, it will be appreciated that “short target nucleic acid” can refer to the short target nucleic acid itself, as well as surrogates thereof, for example amplification products, and native sequences. In some embodiments, the short target nucleic is a short DNA molecule derived from a degraded source, such as can be found in for example but not limited to forensics samples (see for example Butler, 2001, Forensic DNA Typing: Biology and Technology Behind STR Markers. The short target nucleic acid of the present teachings can be derived from any of a number of sources, including without limitation, viruses, prokaryotes, eukaryotes, for example but not limited to plants, fungi, and animals. These sources may include, but are not limited to, whole blood, a tissue biopsy, lymph, bone marrow, amniotic fluid, hair, skin, semen, biowarfare agents, anal secretions, vaginal secretions, perspiration, saliva, buccal swabs, various environmental samples (for example, agricultural, water, and soil), research samples generally, purified samples generally, cultured cells, and lysed cells. It will be appreciated that short target nucleic acids can be isolated from samples using any of a variety of procedures known in the art, for example the Applied Biosystems ABI Prism™ 6100 Nucleic Acid PrepStation, and the ABI Prism™ 6700 Automated Nucleic Acid Workstation, Boom et al., U.S. Pat. No. 5,234,809, mirVana RNA isolation kit (Ambion), etc. It will be appreciated that polynucleotides can be cut or sheared prior to analysis, including the use of such procedures as mechanical force, sonication, restriction endonuclease cleavage, or any method known in the art, to produce short target nucleic acids. In general, the short target nucleic acids of the present teachings will be single stranded, though in some embodiments the short target nucleic can be double stranded, and/or comprise double-stranded regions due to secondary structure, and a single strand can result from denaturation

As used herein, the term “reverse transcription reaction” refers to an elongation reaction in which the 3′ target-specific portion of a target-specific stem-loop reverse transcription primer, and the oligo-dT end of the oligo-dT-promoter-containing reverse transcription primer, are extended to form an extension reaction product comprising complementary strands to the short target nucleic acids and the messenger RNAs. In some embodiments, the short target nucleic acid is a miRNA molecule and the extension reaction is a reverse transcription reaction comprising a reverse transcriptase, where the 3′ end of a target-specific stem-loop reverse transcription primer is extended. In some embodiments, the extension reaction is a reverse transcription reaction comprising a polymerase derived from a Eubacteria. In some embodiments, the extension reaction can comprise rTth polymerase, for example as commercially available from Applied Biosystems catalog number N808-0192, and N808-0098.

In some embodiments, the short target nucleic acid is a DNA molecule and the extension reaction comprises a polymerase and results in the synthesis of a complementary strand of DNA. The term reverse transcription can thus also include the synthesis of a DNA complement of a template DNA molecule, as well as the synthesis of a DNA complement of a template RNA molecule.

As used herein, the term “hybridization” refers to the complementary base-pairing interaction of one nucleic acid with another nucleic acid that results in the formation of a duplex, triplex, or other higher-ordered structure, and is used herein interchangeably with “annealing.” Typically, the primary interaction is base specific, e.g., A/T and G/C, by Watson/Crick and Hoogsteen-type hydrogen bonding. Base-stacking and hydrophobic interactions can also contribute to duplex stability. Conditions for hybridizing primers to complementary and substantially complementary target sequences are well known, e.g., as described in Nucleic Acid Hybridization, A Practical Approach, B. Hames and S. Higgins, eds., IRL Press, Washington, D.C. (1985) and J. Wetmur and N. Davidson, Mol. Biol. 31:349 et seq. (1968). In general, whether such annealing takes place is influenced by, among other things, the length of the polynucleotides and the complementary, the pH, the temperature, the presence of mono- and divalent cations, the proportion of G and C nucleotides in the hybridizing region, the viscosity of the medium, and the presence of denaturants. Such variables influence the time required for hybridization. Thus, the preferred annealing conditions will depend upon the particular application. Such conditions, however, can be routinely determined by the person of ordinary skill in the art without undue experimentation. It will be appreciated that complementarity need not be perfect; there can be a small number of base pair mismatches that will minimally interfere with hybridization between the target sequence and the primers of the present teachings. However, if the number of base pair mismatches is so great that no hybridization can occur under minimally stringent conditions then the sequence is generally not a complementary target sequence. Thus, complementarity herein is meant that primers are sufficiently complementary to the target sequence to hybridize under the selected reaction conditions to achieve the ends of the present teachings. Likewise, the immobilized probes on the solid support are sufficiently complementary to the in vitro transcription products to hybridize under the selected reaction conditions to achieve the ends of the present teachings.

The term “corresponding” as used herein refers to a specific relationship between the elements to which the term refers. Some non-limiting examples of corresponding include: a stem-loop reverse transcription primer can correspond to a particular short target nucleic acid, the in vitro transcription product of a particular species of short target nucleic acid can correspond to a particular immobilized probe on a solid support, etc.

As used herein, the term “detection” refers to the application of any of a variety of microarrays, labeling procedures, and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:140-45, including supplements, 2003). It will also be appreciated that detection can comprise reporter groups that are incorporated into the reaction products, for example due to the incorporation of labeled dNTPs during an in vitro amplification, or attached to reaction products, for example but not limited to the inclusion DIG-labeled dUTP (Digoxigenin-labeled dUTP) in the reaction, with subsequent labeling with alkaline-phosphatase-based chemiluminescence. Some illustrative detection methods are further described in U.S. Pat. No. 6,905,826.

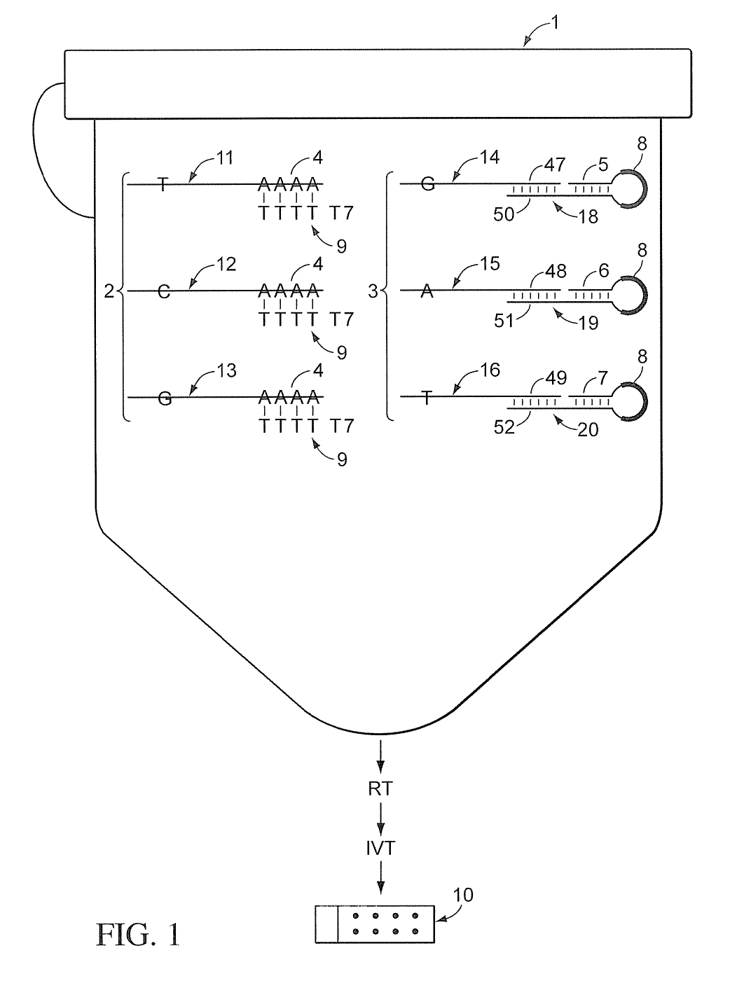

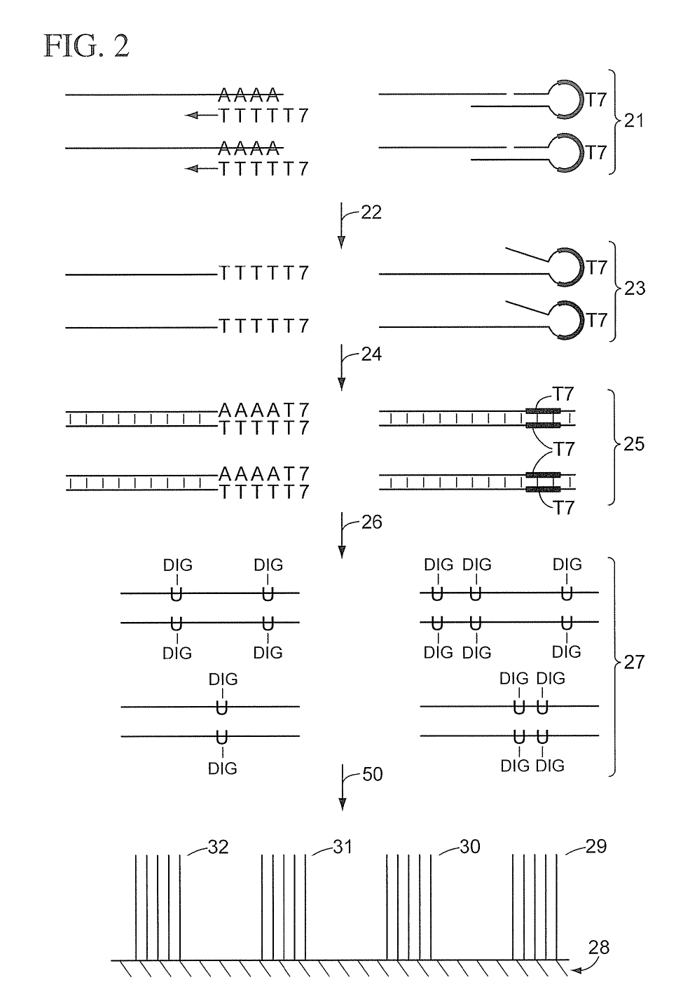

As used herein, the term “target-specific stem-loop reverse transcription primer” refers to a molecule comprising a 3′ target specific portion, a stem, and a loop. Illustrative target-specific stem-loop reverse transcription primers are depicted in FIG. 1 (molecules 18, 19, and 20), elsewhere in the present teachings in FIG. 2, and in U.S. patent application Ser. No. 10/947,460 to Chen et al., and U.S. patent application Ser. No. 11/421,449 to Lao et al., The term “3′ target-specific portion” refers to the single stranded portion of the target-specific stem-loop reverse transcription primer that is complementary to a short target nucleic, such as a micro RNA or endogenous control small RNA. The 3′ target-specific portion is located downstream from the stem of the target-specific stem-loop reverse transcription primer. Generally, the 3′ target-specific portion is between 6 and 9 nucleotides long. In some embodiments, the 3′ target-specific portion is 7 nucleotides long. It will be appreciated that routine experimentation can produce other lengths, and that 3′ target-specific portions that are longer than 8 nucleotides or shorter than 6 nucleotides are also contemplated by the present teachings. In some embodiments, modified bases such as LNA can be used in the 3′ target specific portion to increase the Tm of the stem-loop primer (see for example Petersen et al., Trends in Biochemistry (2003), 21:2:74-81). In some embodiments, universal bases can be used, for example to allow for smaller libraries of stem-loop primers. In some embodiments, modifications including but not limited to LNAs and universal bases can improve reverse transcription specificity and potentially enhance detection specificity. The term “stem” refers to the double stranded region of the target-specific stem-loop reverse transcription primer that is between the 3′ target-specific portion and the loop, and is discussed more fully below. The term “loop” refers to a region of the stem-loop primer that is located between the two complementary strands of the stem, as depicted for example in FIG. 1 (labeled as (8)). Typically, the loop comprises single stranded nucleotides, though other moieties including modified DNA or RNA, Carbon spacers such as C18, and/or PEG (polyethylene glycol) are also possible. Generally, the loop is between 4 and 30 nucleotides long. In some embodiments, the loop is between 14 and 18 nucleotides long. In some embodiments, the loop is 16 nucleotides long. Those in the art will appreciate that loops shorter that 4 nucleotides and longer than 20 nucleotides can be identified in the course of routine methodology and without undue experimentation, and that such shorter and longer loops are contemplated by the present teachings. Generally, the loop will be at least long enough to include sufficient sequence information to encode a promoter. In some embodiments, some of the sequence information encoding the promoter can also reside in the stem. In some embodiments, a target-specific reverse transcription primer can be employed that lacks a stem-loop structure. Such linear primers can contain a 3′ target-specific portion, with sequence information encoding a zip-code and a promoter upstream (5′ of) the target-specific portion.

As used herein, the term “zip-code stem” refers to the double-stranded region of the target-specific stem-loop reverse transcription primer. The stem of the target-specific stem-loop reverse transcription primer can be 8 base-pairs in length. In some embodiments, the stem can be 9 base-pairs in length. In some embodiments, the stem can be 10 base-pairs in length. In some embodiments, the stem can be 11 base-pairs in length. In some embodiments, the stem can be 12 base-pairs in length. In some embodiments, the stem can be 13 base-pairs in length. In some embodiments, the stem can be 14 base-pairs in length. Generally, longer stems are possible, but will come at the cost of increased expense in oligonucleotide manufacturing, and will further add to reaction complexity. In some embodiments, the stem can be 7 base-pairs in length. In some embodiments, the stem can be 6 base-pairs in length. Stems shorter than 6 base-pairs in length are possible, but are done at the sacrifice of specificity at the level of binding to the immobilized probe on an array. Generally, one of the complementary strands of the stem can comprise an identifying portion, referred to herein as a “zip-code.” Descriptions of zip-codes can be found in, among other places, U.S. Pat. Nos. 6,309,829 (referred to as “tag segment” therein); 6,451,525 (referred to as “tag segment” therein); 6,309,829 (referred to as “tag segment” therein); 5,981,176 (referred to as “grid oligonucleotides” therein); 5,935,793 (referred to as “identifier tags” therein); and PCT Publication No. WO 01/92579 (referred to as “addressable support-specific sequences” therein).

As used herein, one can distinguish between the two complementary strands of the stem by the terms “3′ stem region of the target-specific stem-loop reverse transcription primer,” which refers to the strand nearest to the 3′ end of the stem-loop reverse transcription primer. The other stand can be referred to as the “5′ stem region of the stem-loop reverse transcription primer.”

### EXEMPLARY EMBODIMENTS

FIG. 1 depicts certain compositions according to some embodiments of the present teachings. Here, a reaction vessel (1) is shown containing a plurality of different messenger RNA species (2, wherein the T, C, and G of molecules 11, 12, and 13, respectively, indicate one of any number of sequence differences between the three species), and a plurality of different short target nucleic acids such as micro RNAs (3, wherein the G, A, and T of molecules 14, 15, and 16, respectively, indicate one of any number of sequence differences between the three species). The plurality of messenger RNAs can hybridize to an oligo-dT-promoter-containing reverse transcription primer (9). Due to the poly-A tail of mature messenger RNA (4), an oligo-dT-promoter-containing reverse transcription primer (9) can be employed to query the diversity of different messenger RNA species with a single species of oligo-dT-promoter-containing reverse transcription primer. The plurality of micro RNAs (3), on the other hand, hybridize to a particular target-specific stem-loop reverse transcription primer. That is, each species of micro RNA (14, 15, and 16), each with their own distinct 3′ end sequence (47, 48, 49), can hybridize to a particular stem-loop reverse transcription primer. For example, the 3′ target-specific portion (50) of micro RNA (14) hybridizes with stem-loop reverse transcription primer (18) by virtue of its distinct 3′ end sequence (47), whereas the 3′ target-specific portion (51) of micro RNA (15) hybridizes with stem-loop reverse transcription primer (19) by virtue of its distinct 3′ end sequence (48), and the 3′ target-specific portion (52) of micro RNA (16) hybridizes with stem-loop reverse transcription primer (20) by virtue of its distinct 3′ end sequence (49). Each species of stem-loop reverse transcription primer comprises a distinct zip-code encoded, for example, in their respective stems. Thus, stem-loop reverse transcription primer (18) comprises a zip-code (5), stem-loop reverse transcription primer (19) comprises a zip-code (6), and stem-loop reverse transcription primer (20) comprises a zip-code (7). Therefore, the resulting reverse transcription product for a given micro RNA species will be encoded with a distinct zip-code. The stem-loop reverse transcription primers further comprises a loop, which here is shown containing a promoter such as T7 (8). Following reverse transcription of the messenger RNAs and the micro RNAs in the same reaction vessel, an in vitro transcription reaction can be performed using T7 polymerase. This in vitro transcription reaction can comprise a labeling reagent, such as DIG-dUTP, thereby allowing for the detection of the resulting in vitro transcription products on a solid support such as an array (10) by the use of conventional visualization approaches such as chemiluminescence.

FIG. 2 depicts some of the aspects of a reaction scheme according to some embodiments of the present teachings. Here, a hybridization reaction (21) comprising short target nucleic acids, messenger RNAs, target-specific stem-loop reverse transcription primers, and an oligo-dT-promoter-containing reverse transcription primer can be followed by an extension reaction (22) to produce a collection of reverse transcription products (23). Thereafter, a second strand synthesis (24) can be performed, thus resulting in a collection of double stranded products (25). Thereafter, an in vitro transcription reaction (26) can be performed in which DIG-labed dUTP is incorporated, to form a collection of labeled in vitro transcription products (27). The labeled in vitro transcription products can be hybridized (50) on a solid support such as an array (28), wherein the array contains a collection of spots (29, 30, 31, 32). Each spot contains several probe molecules. Additional teachings describing array spots, and the use of internal control polynucleotides, can be found in U.S. Pat. No. 6,905,826.

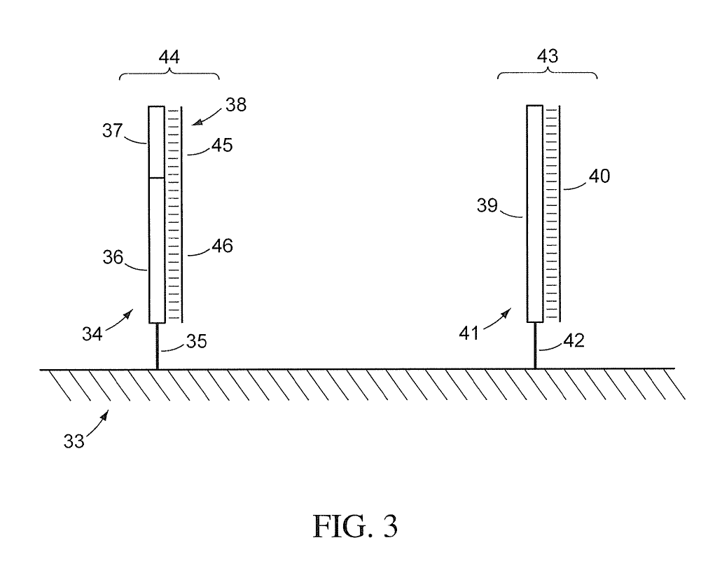

FIG. 3 depicts two different probes (34 and 41) found in two different spots (43 and 44) on the solid support (33). Here, a probe (41) for a messenger RNA can contain a sequence portion (39) complementary to the messenger RNA, or complementary to the complement of the messenger RNA. The probe (41) can be attached to the solid support with a linker moiety (42). See U.S. Pat. No. 7,083,917 and U.S. Pat. No. 6,852,487 for illustrative linker moieties, as well as illustrative array approaches. The corresponding in vitro transcription product for the messenger RNA (40) can hybridize to the sequence portion (39) of the probe (41) by Watson-Crick hydrogen bonding. Also shown in FIG. 3 is a probe (34) for a short target nucleic acid, which can contain a sequence portion (36) complementary to the short target nucleic acid, or complementary to the complement of the short target nucleic acid. The probe (34) further contains the sequence of the zip-code, or a sequence complementary to the zip-code (37), that was introduced by the stem of the target-specific stem-loop reverse transcription primer in the reverse transcription reaction. Thus, the corresponding in vitro transcription product for the short target nucleic acid (38) can hybridize to the sequence portions of the probe (36 and 37) by Watson-Crick hydrogen bonding. The in vitro transcription product for the corresponding short target nucleic acid (38) has a portion corresponding to the short target nucleic acid (46), and a portion corresponding to the zip-code that was introduced by the stem of the target-specific stem-loop reverse transcription primer (45). Thus, (37) can hybridize to (45), and (36) can hybridize to (46). It will be appreciated that a plurality molecules can exist at each spot (43 and 44) on the solid support, thus allowing for several molecules of a labeled amplified messenger RNA to hybridize to spot (43), and several molecules of a labeled amplified short target nucleic acid to hybridize to spot (44), (and also see FIG. 2). The length of the immobilized probes can vary according to the experimentalist. In some embodiments, the probes are 60 residues in length. In embodiments, the probes are 58-62 residues in length. In some embodiments, the probes are spotted on the array surface. In some embodiments the probes are synthesized on the array surface, for example using photolithography, though typically probes will be shorter when synthesized using photolithography to avoid unwanted truncation products.

Thus, in some embodiments the present teachings provide a method of detecting a plurality of short target nucleic acids, and a plurality of messenger RNAs on a same solid support, wherein each short target nucleic acid is 18-25 nucleotides in length, said method comprising; contacting the plurality of different short target nucleic acids with a plurality of target-specific stem-loop reverse transcription primers, wherein each of the plurality of stem-loop reverse transcription primers comprises a 3′ target-specific portion, a zip-code stem, and a promoter; contacting, in the same reaction mixture as the short target nucleic acids, a plurality of messenger RNAs with an oligo-dT-promoter-containing reverse transcription primer; extending the plurality of target-specific reverse transcription primers and the oligo-dT-promoter-containing reverse transcription primer in a reverse transcription reaction to form a collection of reverse transcription products; amplifying the reverse transcription products in an in vitro transcription reaction comprising an enzyme corresponding to the promoter, to form a plurality of in vitro transcription products; and, detecting the plurality of in vitro transcription products on the same solid support. In some embodiments, the plurality of short target nucleic acids are micro RNAs. In some embodiments, the loop of the target-specific stem-loop reverse transcription primers comprises the promoter. In some embodiments, the promoter is T7, though any of a variety of other promoters may be employed, including for example SP6 and T3. Methods employing promoter sequences to effectuate in vitro transcription are known, and can be found for example in U.S. Pat. No. 5,514,545, U.S. Pat. No. 5,545,522, U.S. Pat. No. 5,554,552, U.S. Pat. No. 5,716,785, U.S. Pat. No. 5,891,636, and U.S. Pat. No. 6,114,152. In some embodiments, the in vitro transcription reaction comprises DIG-dUTP, though any of a variety of labeling means can be employed, including the amino-allyl labeling procedure. In some embodiments, the plurality of short target nucleic acids comprises at least 100 different short target nucleic acid species. In some embodiments, the plurality of short target nucleic acids comprises at least 200 different short target nucleic acid species. In some embodiments, the plurality of short target nucleic acids comprises at least 300 different short target nucleic acid species.

In some embodiments, the present teachings provide a reaction composition comprising;

(A) a plurality of target-specific stem-loop reverse transcription primers, wherein each of the plurality of target-specific stem-loop reverse transcription primers comprises a 3′ target-specific portion, a zip-code stem, a promoter; and,

(B) an oligo-dT-promoter-containing reverse transcription primer. In some embodiments, the plurality of target-specific stem-loop reverse transcription primers comprise at least 100 target-specific stem-loop reverse transcription primer species. In some embodiments, the plurality of target-specific stem-loop reverse transcription primers comprise at least 200 target-specific stem-loop reverse transcription primers. In some embodiments, the plurality of target-specific stem-loop reverse transcription primers comprise at least 300 target-specific stem-loop reverse transcription primers. The number of T residues in the oligo-dT-promoter-containing reverse transcription primer can vary, and in some embodiments includes at least 5, at least 10, at least 15, at least 20, and greater than 20 T residues.

In some embodiments, the present teachings provide a device comprising a solid support comprising a plurality of immobilized probes, wherein the plurality of immobilized probes comprises; A) a plurality of probes complementary to, or complementary to the complement of, the plurality of messenger RNAs; and, B) a plurality of probes complementary to, or complementary to the complement of, the plurality of short target nucleic acids and the corresponding zip-code introduced in a reverse transcription reaction. In some embodiments, the immobilized probes comprise PNA. In some embodiments, the immobilized probes comprise DNA. In some embodiments, various analogs can be employed, as discussed supra. In some embodiments, the plurality of probes complementary to, or complementary to the complement of, the plurality of messenger RNAs comprise at least 1000 different probes. In some embodiments, the plurality of probes complementary to, or complementary to the complement of, the plurality of messenger RNAs comprise probes for an entire transcriptome. In some embodiments, the plurality of probes complementary to, or complementary to the complement of, the plurality of short target nucleic acids and the corresponding zip-code introduced in the reverse transcription reaction comprise at least 100 different probes.

### Kits

In certain embodiments, the present teachings also provide kits designed to expedite performing certain methods. In some embodiments, kits serve to expedite the performance of the methods of interest by assembling two or more components used in carrying out the methods. In some embodiments, kits may contain components in pre-measured unit amounts to minimize the need for measurements by end-users. In some embodiments, kits may include instructions for performing one or more methods of the present teachings. In certain embodiments, the kit components are optimized to operate in conjunction with one another.

Thus, in some embodiments the present teachings provide a kit for quantitating a plurality of short target nucleic acids, and a plurality of messenger RNAs on a single solid support, wherein each short target nucleic acid is 18-25 nucleotides in length, said method comprising; A) a plurality of target-specific stem-loop reverse transcription primers, wherein each of the plurality of target-specific stem-loop reverse transcription primers comprises a 3′ target-specific portion, a zip-code stem, and a promoter; and, B) an oligo-dT-promoter-containing reverse transcription primer. In some embodiments, the kit further comprises a reverse transcriptase. In some embodiments, the kit further comprises dNTPs.

In some embodiments, the kit further comprises DIG-dUTP. In some embodiments, the kit further comprises a solid support, wherein the solid support comprises a plurality of immobilized probes, wherein the plurality of immobilized probes comprise; A) a plurality of probes complementary to, or complementary to the complement of, the plurality of messenger RNAs; and, B) a plurality of probes complementary to, or complementary to the complement of, the plurality of short target nucleic acids and the corresponding zip-code introduced in a reverse transcription reaction. In some embodiments, the kit further comprises immobilized probes that comprise PNA. In some embodiments, the kit further comprises immobilized probes that comprise DNA.

While the present teachings have been described in terms of these exemplary embodiments, the skilled artisan will readily understand that numerous variations and modifications of these exemplary embodiments are possible without undue experimentation. All such variations and modifications are within the scope of the current teachings. Aspects of the present teachings may be further understood in light of the following example, which should not be construed as limiting the scope of the teachings in any way.

Although the disclosed teachings have been described with reference to various applications, methods, kits, and compositions, it will be appreciated that various changes and modifications may be made without departing from the teachings herein and the claimed invention below. The foregoing examples are provided to better illustrate the disclosed teachings and are not intended to limit the scope of the teachings presented herein.

1. A method of detecting a plurality of short target nucleic acids, and a plurality of messenger RNAs on a same solid support, wherein each short target nucleic acid is 18-25 nucleotides in length, said method comprising;
contacting the plurality of different short target nucleic acids with a plurality of target-specific stem-loop reverse transcription primers, wherein each of the plurality of stem-loop reverse transcription primers comprises a 3′ target-specific portion, a zip-code stem, and a promoter;
contacting, in the same reaction mixture as the short target nucleic acids, a plurality of messenger RNAs with an oligo-dT-promoter-containing reverse transcription primer;
extending the plurality of reverse transcription primers and the oligo-dT-promoter-containing reverse transcription primer in a reverse transcription reaction to form a collection of reverse transcription products;
amplifying the reverse transcription products in an in vitro transcription reaction comprising an enzyme corresponding to the promoter, to form a plurality of in vitro transcription products; and,
detecting the plurality of in vitro transcription products on the same solid support.

2. The method according to claim 1 wherein the plurality of short target nucleic acids are micro RNAs.

3. The method according to claim 1 wherein the loop of the target-specific stem-loop reverse transcription primers comprises the promoter.

4. The method according to claim 1 wherein the promoter is T7.

5. The method according to claim 1 wherein the in vitro transcription reaction comprises DIG-dUTP.

6. The method according to claim 1 wherein the plurality of short target nucleic acids comprises at least 100 different short target nucleic acid species.

7. A reaction composition comprising;
(A) a plurality of target-specific stem-loop reverse transcription primers, wherein each of the plurality of target-specific stem-loop reverse transcription primers comprises a 3′ target-specific portion, a zip-code stem, a promoter; and,
(B) an oligo-dT-promoter-containing reverse transcription primer.

8. The reaction composition according to claim 7 wherein the plurality of target-specific stem-loop reverse transcription primers comprises at least 100 target-specific stem-loop reverse transcription primer species.

9. A kit for quantitating a plurality of short target nucleic acids, and a plurality of messenger RNAs on a single solid support, wherein each short target nucleic acid is 18-25 nucleotides in length, said method comprising;
A) a plurality of target-specific stem-loop reverse transcription primers, wherein each of the plurality of target-specific stem-loop reverse transcription primers comprises a 3′ target-specific portion, a zip-code stem, and a promoter; and,
B) an oligo-dT-promoter-containing reverse transcription primer.

10. The kit according to claim 9 further comprising a reverse transcriptase.

11. The kit according to claim 9 further comprising dNTPs.

12. The kit according to claim 9 further comprising DIG-dUTP.

13. The kit according to claim 9 further comprising a solid support, wherein the solid support comprises a plurality of immobilized probes, wherein the plurality of immobilized probes comprises;
A) a plurality of probes complementary to, or complementary to the complement of, a plurality of messenger RNAs; and,
B) a plurality of probes complementary to, or complementary to the complement of, a plurality of short target nucleic acids and a corresponding zip-code introduced in a reverse transcription reaction.

14. The kit according to claim 13 wherein the immobilized probes comprise PNA.

15. The kit according to claim 13 wherein the immobilized probes comprise DNA.

16. A device comprising a solid support comprising a plurality of immobilized probes, wherein the plurality of immobilized probes comprises;
A) a plurality of probes complementary to, or complementary to the complement of, a plurality of messenger RNAs; and,
B) a plurality of probes complementary to, or complementary to the complement of, a plurality of short target nucleic acids and a corresponding zip-code introduced in a reverse transcription reaction.

17. The device according to claim 16 wherein the immobilized probes comprise PNA.

18. The device according to claim 16 wherein the immobilized probes comprise DNA.

19. The device according to claim 16 wherein the plurality of probes complementary to, or complementary to the complement of, the plurality of messenger RNAs comprise at least 1000 different probes.

20. The device according to claim 16 wherein the plurality of probes complementary to, or complementary to the complement of, the plurality of short target nucleic acids and the corresponding zip-code introduced in the reverse transcription reaction comprise at least 100 different probes.
